(19) United States
(12) Patent Application Publication
(10) Pub. No.: US 2006/0276812 A1
(43) Pub. Date: Dec. 7, 2006

(54) Dynamic reinforcement of the lower esophageal sphincter

(76) Inventors: James W. Hill, Mission Viejo, CA (US); Shawn Moaddeb, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/396,500

(22) Filed: Apr. 3, 2006

Related U.S. Application Data
(60) Provisional application No. 60/668,040, filed on Apr. 4, 2005.

(51) Int. Cl. A61F 2/02; A61B 17/12

(52) U.S. Cl. 606/157; 623/14.13; 623/23.68; 128/DIG. 25

(57) ABSTRACT

Gastroesophageal implants are implantable at or near the gastroesophageal junction in order reinforce the lower esophageal sphincter and prevent gastric reflux. In a contracted configuration, the implants prevent or substantially restrict communication between the stomach and the esophagus. In an open configuration, the implants do not substantially restrict communication between the stomach and the esophagus. Certain embodiments of the implants are capable of detecting various conditions of the esophagus and/or stomach and moving between the contracted and open configurations in response to the detected condition(s).

### CROSS-REFERENCE TO RELATED APPLICATION

This application claims priority to provisional application Ser. No. 60/668,040, filed on Apr. 4, 2005, the entire contents of which are hereby incorporated by reference.

### BACKGROUND OF THE INVENTION

1. Field of the Invention

The present invention relates to devices and methods for treating gastroesophageal disorders.

2. Description of the Related Art

The lower esophageal sphincter (LES) is a ring-shaped muscle that forms a valve at the junction of the esophagus and the stomach. The LES normally remains closed. However, when one swallows, a food bolus travels downward through the esophagus toward the stomach. When the food bolus reaches the lower end of the esophagus, the LES opens to allow the bolus to pass from the esophagus into the stomach. After the food bolus has passed, the LES again closes. When the LES is closed, it prevents the backflow (reflux) of hydrochloric acid and other gastric contents into the esophagus. If the LES does not close adequately, stomach acid may reflux into the esophagus, causing heartburn. Persistent reflux can lead to Barrett's esophagus, and, in advanced cases, esophageal cancer. A weak or incompetent LES is a major cause of gastroesophageal reflux disease (GERD).

### SUMMARY OF THE INVENTION

The preferred embodiments of the present dynamic reinforcement of the lower esophageal sphincter have several features, no single one of which is solely responsible for their desirable attributes. Without limiting the scope of these implants and methods as expressed by the claims that follow, their more prominent features will now be discussed briefly. After considering this discussion, and particularly after reading the section entitled “Detailed Description of the Preferred Embodiments,” one will understand how the features of the preferred embodiments provide advantages, which include the capability to dynamically reinforce the LES, thereby preventing gastric reflux, while also allowing food to pass through the LES and into the stomach.

One embodiment of the present dynamic reinforcement of the lower esophageal sphincter comprises an implant configured to encompass, at least partially, a portion of a person's gastrointestinal tract at or near the gastroesophageal junction thereof. The implant comprises an implant body, a sensor configured to detect a condition of the person's esophagus, and an actuator coupled to the implant body and in communication with the sensor. The implant is configured to change from a contracted configuration, in which the implant at least partially constricts the gastrointestinal tract at or near the gastroesophageal junction, to an open configuration, in which the implant does not substantially constrict the gastrointestinal tract. The actuator is configured to apply force to the implant body in changing the implant from the open configuration to the contracted configuration, and/or from the contracted configuration to the open configuration, in response to the condition of the esophagus detected by the sensor.

In some embodiments the actuator may be configured to apply a force to the body to cause the body to move from the contracted configuration to the open configuration.

In some embodiments the actuator may be configured to apply a force to the body to cause the body to move from the open configuration to the contracted configuration.

In some embodiments the condition of the person's esophagus may comprise at least one characteristic of an electrical signal emanating from the esophagus.

In some embodiments the condition of the person's esophagus may comprise a pressure and/or at least one characteristic of a pressure wave detected from the esophagus.

In some embodiments the actuator may comprise a motor.

In some embodiments the actuator may further comprise a linear translator.

In some embodiments the actuator may further comprise a power source.

Some embodiments may further comprise a processor in electrical communication with the sensor.

In some embodiments the processor may be configured to receive an input signal from the sensor and to produce an output signal to be transmitted to the actuator.

In some embodiments the actuator may be at least partially contained within the implant body.

In some embodiments the sensor may be configured to measure a frequency pattern and/or an amplitude pattern of peristaltic waves.

In some embodiments the sensor may comprise a pressure sensor, or a strain gauge, or an electrode.

Another embodiment of the present dynamic reinforcement of the lower esophageal sphincter comprises an implant configured to encompass, at least partially, a portion of a human esophagus at or near a lower esophageal sphincter thereof. The implant comprises an implant body, and means for moving the body between a contracted configuration, in which the implant constricts the gastrointestinal tract at or near the gastroesophageal junction, and an open configuration, in which the implant does not substantially constrict the gastrointestinal tract.

Some embodiments may further comprise means for sensing a condition of the person's esophagus, the means for sensing being in communication with the means for moving.

In some embodiments the means for moving the body may comprise a motor and a linear translator.

Another embodiment of the present dynamic reinforcement of the lower esophageal sphincter comprises a method of reinforcing a lower esophageal sphincter of a patient's esophagus. The method comprises the step of securing an implant at or near the lower esophageal sphincter, such that the implant at least partially encompasses a portion of the patient's gastrointestinal tract at or near the gastroesophageal junction, and at least partially constricts the gastrointestinal tract. The method comprises the steps of allowing the implant to sense a condition of the esophagus, and allowing the implant to open in response to the sensed condition such that the implant does not substantially constrict the gastrointestinal tract at or near the gastroesophageal junction.

In some embodiments the method further comprises the step of allowing the implant to constrict the gastrointestinal tract after a predetermined interval.

In some embodiments the method further comprises allowing the implant to constrict the gastrointestinal tract automatically in response to a further sensed condition of the esophagus.

### BRIEF DESCRIPTION OF THE DRAWINGS

The preferred embodiments of the present dynamic reinforcement of the lower esophageal sphincter, illustrating their features, will now be discussed in detail. These embodiments depict the novel and non-obvious implants and methods shown in the accompanying drawings, which are for illustrative purposes only. These drawings include the following figures, in which like numerals indicate like parts:

FIG. 1 is a front elevational view of a human stomach and esophagus, including one embodiment of the present gastric implants;

FIG. 2 is a detail view of the gastroesophageal junction of FIG. 1, including the implant;

FIG. 3 is a cross-sectional view of the gastroesophageal junction of FIG. 2, taken along the line 3-3 of FIG. 2;

FIG. 4 is a cross-sectional view of the gastroesophageal junction of FIG. 3, illustrating the implant in a contracted configuration and the esophagus in a constricted or closed configuration;

FIG. 5 is a front elevational view of a gastroesophageal junction and another embodiment of the present gastric implants;

FIG. 6 is a schematic top plan view of another embodiment of the present gastric implants;

FIG. 7 is a schematic top plan view of another embodiment of the present gastric implants; and

FIG. 8 is a schematic top plan view of another embodiment of the present gastric implants.

### DETAILED DESCRIPTION OF THE PREFERRED EMBODIMENTS

FIG. 1 illustrates a human stomach 20 and esophagus 22, including one embodiment 24 of the present gastric implants. As shown in detail in FIG. 2, the implant 24 is disposed about a lower end of the esophagus 22 near the junction of the esophagus 22 and the stomach 20. The LES is located in this region. As discussed above, a healthy LES provides selective communication between the esophagus and the stomach, thereby allowing food to pass into the stomach as needed, while preventing unwanted reflux of stomach contents. As discussed in detail below, the implant 24 reinforces a weak LES by constricting the lower end of the esophagus 22 to prevent reflux. The implant 24 advantageously senses the state of the esophagus 22 and/or stomach 20 and relaxes at appropriate moments in order to allow food boluses to pass into the stomach 20. When each food bolus has passed, the implant 24 again contracts and restricts communication between the esophagus 22 and the stomach 20.

With reference to FIGS. 3 and 4, in the illustrated embodiment the implant 24 comprises an implant body 26 that is shaped substantially as a partial toroid. FIG. 4 illustrates the implant 24 in one example of a contracted configuration, while FIG. 3 illustrates the implant 24 in one example of an open configuration. In the contracted configuration the implant body 26 is sized and shaped to constrict the lower end of the esophagus 22 and thereby prevent reflux. As illustrated, the esophagus 22 is constricted to a pinpoint sized opening 28 that prevents the passage of most, if not all, stomach contents into the esophagus 22. The implant 24 may, of course, be configured to constrict the esophagus 22 more tightly so that substantially no fluid may pass from the stomach 20 into the esophagus 22.

In the open configuration of FIG. 3, the implant body 26 is sized and shaped to allow the lower end of the esophagus to form an opening 28 of sufficient size to allow food boluses to pass into the stomach. In the illustrated embodiment, the implant body 26 extends approximately four-fifths of the way around the esophagus 22, from a first end 30 to a second end 32. However, those of skill in the art will appreciate that the implant 24 could have any of a variety of shapes. For example, the implant body could extend around a smaller or larger fraction of the esophagus. The implant body could also extend completely around the esophagus and be shaped as a complete toroid having interlocking male and female ends, or be shaped as a coil. The implant 24 may be secured to the esophagus so that it does not migrate to another area of the body. For example, sutures (not shown) may tether the implant to the esophageal tissue, or adhesive (such as methyl methacrylate) may secure the implant to the esophageal tissue.

In some embodiments, when the patient is not swallowing, or when a food bolus is not attempting to pass into the stomach, the implant body 26 is in the constricted configuration of FIG. 4. In this configuration the implant body 26 provides support to the LES, causing the LES to close tightly enough to reduce or eliminate reflux of stomach contents. In some embodiments the implant 24 is capable of detecting one or more conditions of the esophagus 22 and/or stomach 20. Such implants are further capable of transitioning between the contracted and open configurations in response to the detected condition(s). Some such embodiments may include a sensor that detects when the patient swallows or when a food bolus is attempting to pass from the esophagus 22 into the stomach 20. Thus, when the patient swallows, the implant body 26 expands to the open configuration of FIG. 3 to allow the LES to open. Once the food bolus passes, the implant body 26 again contracts. For example, the implant 24 may be configured to automatically contract after a preset interval. Such an interval may be 2 or 3 seconds, for example. Alternatively, the implant may be configured to contract only after peristaltic waves are no longer substantially detected.

In some embodiments the sensor 34 may be positioned on the esophagus 22 and be able to communicate (via appropriate connectors 36, such as electrical, optical, etc.) with the implant body 38, as illustrated in FIG. 5. In other embodiments the sensor 34 may be integrated with the implant body 42, as illustrated in FIGS. 6-8. When the sensor 40 is integrated with the implant body 42, it may be positioned on an inner surface 44 of the implant body 42, as shown. Alternatively, the sensor may be positioned elsewhere on the implant body.

The sensor 34, 40 may sense peristaltic waves in the esophagus 22 when the patient swallows. The sensor 34, 40 may be configured to measure a frequency pattern and/or an amplitude pattern of the peristaltic waves. The implant 38, 46, 48, 50 may then be configured to open when the sensor 34, 40 detects that a frequency threshold and/or an amplitude threshold has been reached. Alternatively, the sensor 34, 40 may comprise a pressure sensor, such as a manometer. A pressure sensor may detect an expansion of the esophagus as a food bolus reaches the portion of the esophagus where the sensor is located. Alternatively, the sensor 34, 40 may comprise a strain gauge that detects when a particular region of the esophagus 22 has expanded (or is attempting to expand) to let a food bolus pass. For example, the strain gauge may be positioned on the esophagus separately from the implant body and communicate (via appropriate connectors, such as electrical, optical, etc.) with the implant body. Alternatively, the strain gauge may be integrated with the implant body. Thus, when a food bolus reaches the portion of the esophagus around which the implant is positioned, the esophagus in that region will attempt to expand, but will be constricted by the implant. The implant may be configured to open slightly under pressure from the expanding esophagus, and the strain gauge may sense the slight relaxation of the implant and trigger a larger relaxation.

In some embodiments the sensor 34, 40 may detect electrical activity of the muscles (e.g., an electromyogram) of the esophagus 22. For example, the sensor 34, 40 may include one or more electrodes that contact the muscle or serosa (outer layer) of the esophagus. In some embodiments the electrode(s) may be inserted into one or more esophageal tissue layers. For example, in the implant 38 of FIG. 5 the sensor 34 may comprise an electrode that has been implanted within the esophageal tissue. Alternatively, in a ring-shaped implant for example, the electrode may be located on an inner surface 44 of the implant body, as with the implants 46, 48, 50 of FIGS. 6-8. When the implants 46, 48, 50 of FIGS. 6-8 are implanted around the esophagus 22, the inward facing sensors 40 contact the esophagus 22. The sensors 34, 40 are preferably configured according to well-known methods so that they accurately detect electrical impulses within the esophageal muscles. For example, the sensors 34, 40 are preferably configured such that noise is reduced.

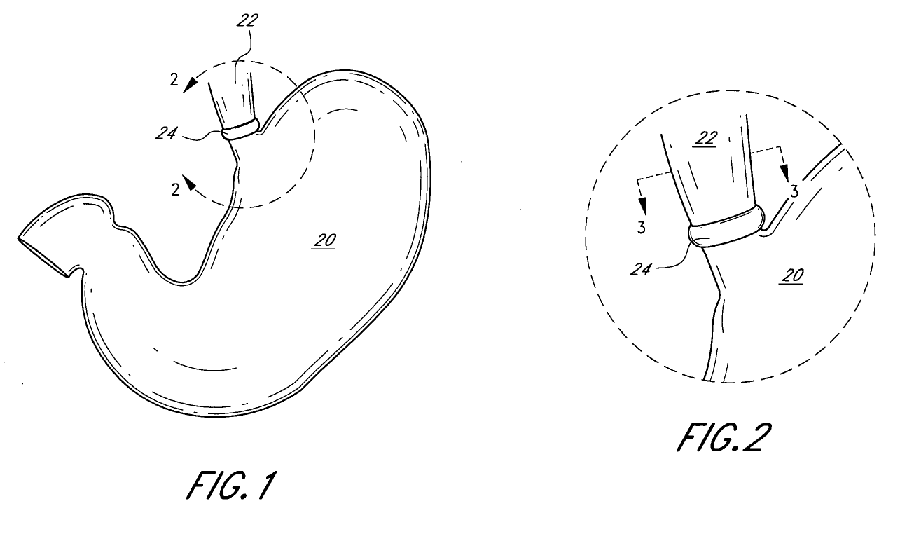

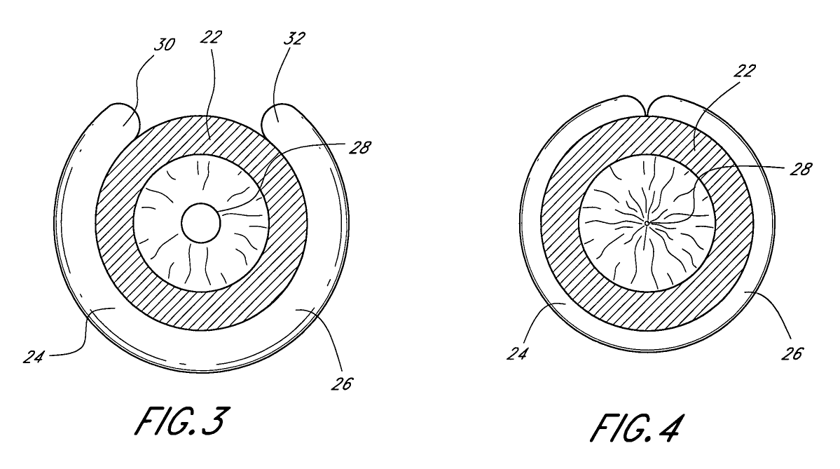

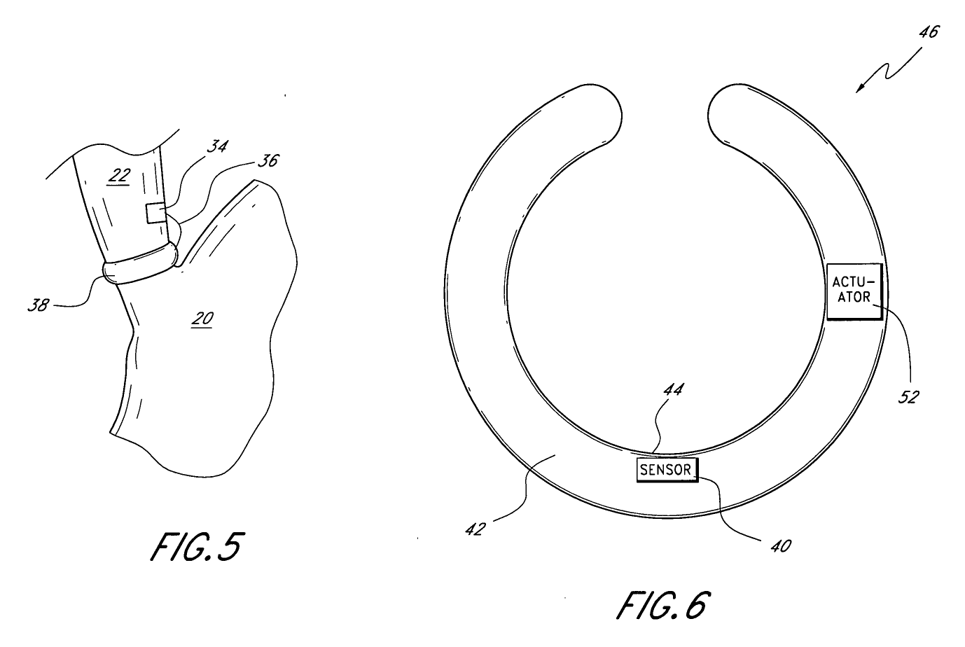

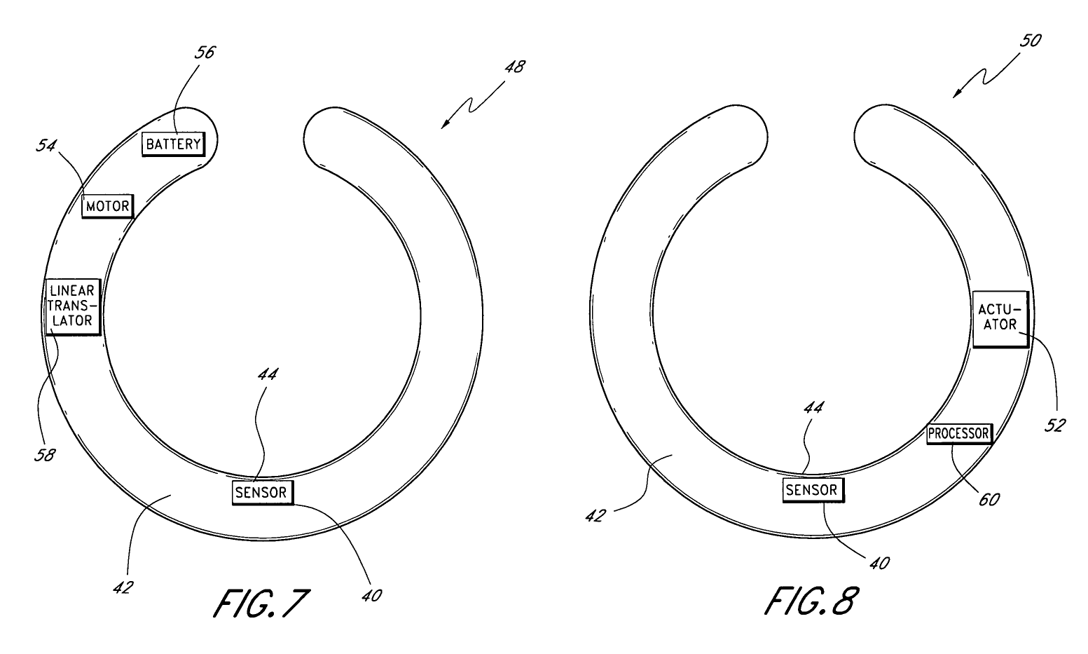

In certain embodiments, the sensor 34, 40 communicates with an actuator 52 (FIGS. 6 and 8) that moves the implant 46, 50 between the open and contracted configurations. In certain of these embodiments the actuator includes one or more motors 54 that are configured to respond to the sensor 40 to relax (open) and contract (close) the implant 48, and a power source 56. For example, FIG. 7 schematically illustrates an implant 48 comprising an implant body 42, a battery 56, a motor 54 and a linear translator 58. The linear translator 58 is configured to resize the implant body 42 in response to signals from the sensor 40. For example, the motor 54, which may be a stepper motor, may provide rotational movement in response to a control signal. The linear translator 58 may then convert the rotational movement of the motor 54 into linear movement. In one embodiment, the linear translator 58 may be coupled to the implant 48 such that activation of the motor 54 causes the linear translator 58 to apply tension to a forming element such as a filament (not shown). Other ways of using a motor to resize the implant can also be used, including, for example, those taught by Lashinski et al. in U.S. Patent Application Publication No. 2005/0060030 A1, which is hereby incorporated by reference.

In some embodiments, a processor 60 communicates with the sensor 40 and with the actuator 52, as illustrated in FIG. 8. The actuator 52 causes the implant body to open upon receiving the appropriate stimulus from the processor 60. The timing of this stimulus can be fine-tuned to coincide properly with the passage of a food bolus through the LES. For example, a clinician may fine-tune the timing of the stimulus by remotely programming the processor 60. One example of a remote programming technique is radiofrequency coupling, which is commonly practiced with cardiac pacemakers and which is well-known to those of skill in the art. In addition or alternatively, fine-tuning of the processor 60 may occur through an automated “learning” process, utilizing artificial intelligence models such as neural networks or fuzzy logic, in ways that are well-known to those of skill in the art.

Those of skill in the art will appreciate that certain components of the present implants could be located externally from the implant body. For example, in one embodiment the motor and the power source could be located in a secondary housing (not shown) that is anchored within the abdominal cavity remote from the implant body. In such an embodiment a coupling (not shown) provides electrical, mechanical, optical, acoustical, magnetic, and/or hydraulic communication between the implant body and the secondary housing. For example, the coupling may comprise a push/pull wire, a flexible rotating shaft, tubing, a control line, a communication line, and/or a power line, depending upon the division of the internal components between the implant and the secondary housing.

### SCOPE OF THE INVENTION

The above presents a description of the best mode contemplated for carrying out the preferred embodiments of the present dynamic reinforcement of the lower esophageal sphincter, and of the manner and process of making and using them, in such full, clear, concise, and exact terms as to enable any person skilled in the art to which they pertain to make and use these dynamic gastric implants and to practice these methods. These implants and methods are, however, susceptible to modifications and alternate constructions from those discussed above that are fully equivalent. Consequently, these implants and methods are not limited to the particular embodiments disclosed. On the contrary, these implants and methods cover all modifications and alternate constructions coming within the spirit and scope of the following claims, which particularly point out and distinctly claim the subject matter of these implants and methods, and equivalents.

What is claimed is:

1. An implant configured to encompass, at least partially, a portion of a person's gastrointestinal tract at or near the gastroesophageal junction thereof, the implant comprising:
an implant body;
a sensor configured to detect a condition of the person's esophagus; and
an actuator coupled to the implant body and in communication with the sensor;
wherein the implant is configured to change from a contracted configuration, in which the implant at least partially constricts the gastrointestinal tract at or near the gastroesophageal junction, to an open configuration, in which the implant does not substantially constrict the gastrointestinal tract; and
wherein the actuator is configured to apply force to the implant body in changing the implant from the open configuration to the contracted configuration, and/or from the contracted configuration to the open configuration, in response to the condition of the esophagus detected by the sensor.

2. The implant of claim 1, wherein the actuator is configured to apply a force to the body to cause the body to move from the contracted configuration to the open configuration.

3. The implant of claim 1, wherein the actuator is configured to apply a force to the body to cause the body to move from the open configuration to the contracted configuration.

4. The implant of claim 1, wherein the condition of the person's esophagus comprises at least one characteristic of an electrical signal emanating from the esophagus.

5. The implant of claim 1, wherein the condition of the person's esophagus comprises a pressure and/or at least one characteristic of a pressure wave detected from the esophagus.

6. The implant of claim 1, wherein the actuator comprises a motor.

7. The implant of claim 6, wherein the actuator further comprises a linear translator.

8. The implant of claim 6, wherein the actuator further comprises a power source.

9. The implant of claim 1, further comprising a processor in electrical communication with the sensor.

10. The implant of claim 9, wherein the processor is configured to receive an input signal from the sensor and to produce an output signal to be transmitted to the actuator.

11. The implant of claim 1, wherein the actuator is at least partially contained within the implant body.

12. The implant of claim 1, wherein the sensor is configured to measure a frequency pattern and/or an amplitude pattern of peristaltic waves.

13. The implant of claim 1, wherein the sensor comprises a pressure sensor, or a strain gauge, or an electrode.

14. An implant configured to encompass, at least partially, a portion of a human esophagus at or near a lower esophageal sphincter thereof, the implant comprising:
an implant body; and
means for moving the body between a contracted configuration, in which the implant constricts the gastrointestinal tract at or near the gastroesophageal junction, and an open configuration, in which the implant does not substantially constrict the gastrointestinal tract.

15. The implant of claim 14, further comprising means for sensing a condition of the person's esophagus, the means for sensing being in communication with the means for moving.

16. The implant of claim 14, wherein the means for moving the body comprises a motor and a linear translator.

17. A method of reinforcing a lower esophageal sphincter of a patient's esophagus, the method comprising the steps of:
securing an implant at or near the lower esophageal sphincter, such that the implant at least partially encompasses a portion of the patient's gastrointestinal tract at or near the gastroesophageal junction, and at least partially constricts the gastrointestinal tract;
allowing the implant to sense a condition of the esophagus; and
allowing the implant to open in response to the sensed condition such that the implant does not substantially constrict the gastrointestinal tract at or near the gastroesophageal junction.

18. The method of claim 17, further comprising the step of allowing the implant to constrict the gastrointestinal tract after a predetermined interval.

19. The method of claim 17, further comprising allowing the implant to constrict the gastrointestinal tract automatically in response to a further sensed condition of the esophagus.
